(19) United States
(12) Patent Application Publication
(10) Pub. No.: US 2016/0244849 A1
(43) Pub. Date: Aug. 25, 2016

(54) MULTIPLEX DIAGNOSTIC ASSAY FOR DETECTING SALMONID PATHOGENS

(71) Applicant: UNIVERSITY OF PRINCE EDWARD ISLAND, Charlottetown (CA)

(72) Inventors: Carmencita V. YASON, Little York (CA); Dante MATEO, Charlottetown (CA); Larry HAMMELL, Charlottetown (CA); Ian GARDNER, Charlottetown (PE)

(73) Assignee: UNIVERSITY OF PRINCE EDWARD ISLAND, Charlottetown, PE (CA)

(21) Appl. No.: 15/028,334

(22) PCT Filed: Oct. 8, 2014
(86) PCT No.: PCT/CA2014/050972

§ 371 Date: Apr. 8, 2016

Related U.S. Application Data
(60) Provisional application No. 61/888,336, filed on Oct. 8, 2013.

(51) Int. Cl. C12Q 1/70; C12Q 1/68

(52) U.S. Cl. CPC C12Q 1/701; C12Q 1/689; C12Q 2600/16; C12Q 2600/158

(57) ABSTRACT

A method is provided for detecting the presence or absence of salmonid pathogens, including Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), and Renibacterium salmoninarum. The method includes steps which may be carried out using a variety of analytical techniques, such as multiplexing RT-PCR, Target Specific Primer Extension (TSPE), and fluidic bead-based technology. PCR primers and TSPE primers which are components of the multiplex diagnostic assay using fluidic bead-based technology for detection of salmonid pathogens are also described.

### FIELD OF INVENTION

The present invention relates generally to a method for the detection of salmonid pathogens in a sample. More specifically, the present invention relates to a molecular diagnostic assay for detecting more than one salmonid pathogen in a sample simultaneously, for example using fluidic bead-based technology and a multiplexed PCR platform.

### BACKGROUND OF THE INVENTION

Viral and bacterial diseases are a major problem in the aquaculture industry. Outbreaks of infectious disease are a challenge facing fish farming operations, including those involving dense populations of fish in the open sea (Robertson, B. “Can we get the upper hand on viral diseases in aquaculture of Atlantic salmon?” Aquaculture Research, 42, 125-131, 2011). Viral and bacterial infections can have severe effects on the fish farming industry, and the importance of prevention, detection, and treatment of outbreaks is well-recognized.

Infectious Hematopoietic Necrosis Virus (IHNV), Infectious Pancreatic Virus (IPNV), Infectious Salmon Anemia Virus (ISAV), Salmon Alphaviruses (SAV) and Viral Hemorrhagic Septicemia Virus (VHSV) are 5 of the most globally detrimental and economically damaging salmonid viruses. IHNV, IPNV, ISAV, SAV, and VHSV are RNA viruses. RNA viruses such as these cause the highest ecological and socio-economical impacts due to disease in European farmed finfish (Gomez-Casado, E.; Estepa, A.; Coll, J. M. “A comparative review of European-farmed finfish RNA viruses and their vaccines” Vaccine, 29(15), 2657-2671, 2011). Clinical and/or postmortem disease diagnosis of these viruses in fish is highly important, especially during disease outbreaks. Some countries have mandated inspections of artificially propagated fish for the presence of these types of fish pathogens as part of programs to limit fish exposure (Williams, K.; Blake, S.; Sweeny, A.; Singer, J. T.; Nicholson, B. L. “Multiplex Reverse Transcriptase PCR Assay for Simultaneous Detection of Three Fish Viruses” Journal of Clinical Microbiology, 37(12), 4139-4141, 1999).

Renibacterium salmoninarum is a gram-positive bacteria that causes bacterial kidney disease (BKD) in salmon. Bacterial kidney disease is a major cause of morbidity and mortality in salmon, and so R. salmoninarum is another economically and environmentally important salmonid pathogen. The detection of R. salmoninarum through the traditional methods (i.e. media culture and immunogical testing) requires a long time, and these methods are not sensitive enough to detect carrier fish. PCR and nested PCR has demonstrated a better sensitivity (Toranzo, A. E.; Magariños, B.; Romalde J. L. “A review of the main bacterial fish diseases in mariculture systems”. Aquaculture 246; 37-61, 2005). Quantitative PCR (qPCR) has also been developed, however, there is no significant difference in sensitivity between qPCR and nPCR (Elliot D. G; Applegate, L. J.; Murray, A. L.; Purcell, M. K.; McKibben, C. L. “Bench-top validation testing of selected immunological and molecular Renibacterium salmoninarum diagnostic assays by comparison with quantitative bacteriological culture”. Journal of Fish Disease 36(9): 779-809, 2013).

Detection and monitoring of IHNV, IPNV, ISAV, SAV, and VHSV is currently done using PCR-based assays and/or Virus Isolation. PCR and bacterial isolation are currently used for detection of R. salmoninarum. Traditional polymerase chain reaction (PCR) and real time PCR both detect a single target at a time in one sample. This can be time consuming and costly, and thus there is a need for more efficient detection methods.

Multiplex PCR techniques have been developed for detection of various pathogens, including agricultural (US Patent Application No. 2011/0070586) and fish (Williams, K. et al. (1999) Journal of Clinical Microbiology. 37(12), 4139-4141) pathogens. However, continued improvement and development is still needed in order to develop a more robust multiplexing assay for salmonid pathogen detection.

### SUMMARY OF THE INVENTION

It is an object of the invention to provide a method for detecting salmonid pathogens in a test sample.

It is also an object of the invention to provide reagents to use in the detection assay, including PCR primers and TSPE primers specific for pathogens such as Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), or Renibacterium salmoninarum.

Accordingly, there is provided herein a method for detecting the presence or absence of at least one pathogen including: IHNV, IPNV, ISAV, SAV VHSV, or Renibacterium salmoninarum in a sample. The method comprises:
PCR amplification of one or more genomic regions of the at least one pathogen using one or more pair of amplification primers,
target specific primer extension (TSPE) templated on the amplified pathogen genomic region(s) using one or more TSPE primer to produce at least one TSPE primer extension product, and
detection of at least one TSPE primer extension product, wherein the presence of at least one TSPE primer extension product indicates the presence of the at least one pathogen;
wherein the one or more pair of primers comprise at least one of the following:
at least one primer for the detection of IHNV selected from an isolated nucleic acid having at least 80% identity to the sequence of SEQ ID NO: 4 or 9, or fragments thereof of at least 15 contiguous nucleotides;
at least one primer for the detection of IPNV, selected from an isolated nucleic acid having at least 80%/6 identity to the sequence of SEQ ID NO: 2 or 7, or fragments thereof of at least 15 contiguous nucleotides;
at least one primer for the detection of ISAV, selected from an isolated nucleic acid having at least 80% identity to the sequence of SEQ ID NO: 1 or 6, or fragments thereof of at least 15 contiguous nucleotides;
at least one primer for the detection of SAV, selected from an isolated nucleic acid having at least 80% identity to the sequence of SEQ ID NO: 5 or 10, or fragments thereof of at least 15 contiguous nucleotides;
at least one primer for the detection of VHSV, selected from an isolated nucleic acid having at least 80% identity to the sequence of SEQ ID NO: 3 or 8, or fragments thereof of at least 15 contiguous nucleotides; and
at least one primer for the detection of Renibacterium salmoninarum selected from an isolated nucleic acid having at least 80% identity to the sequence of SEQ ID NO: 16 or 17, or fragments thereof of at least 15 contiguous nucleotides.

There is also herein provided a method for detecting the presence or absence of at least one pathogen including: IHNV, IPNV, ISAV, SAV, VHSV, or Renibacterium salmoninarum in a sample. The method comprises:
PCR amplification of one or more genomic regions of the at least one pathogen using one or more pair of primers,
target specific primer extension (TSPE) templated on the amplified pathogen genomic region(s) using one or more TSPE primer to produce at least one TSPE primer extension product, and
detection of the TSPE primer extension products, wherein the presence of at least one of the TSPE primer extension products indicates the presence of the at least one pathogen;
wherein the one or more pathogen genomic regions comprise at least one of the following:
a pathogen genomic region for the detection of IHNV that is within or near the IHNV glycoprotein genomic region (GenBank Acc. No. AY331666) within the IHNV genomic sequence;
a pathogen genomic region for the detection of IPNV that is within or near the IPNV Major capsid polypeptide VP2 genomic region (GenBank Acc. No. FN257526) within the IPNV genomic sequence;
a pathogen genomic region for the detection of ISAV that is within or near the ISAV Segment 8 genomic region (GenBank Acc. No. AF404340) within the ISAV genomic sequence;
a pathogen genomic region for the detection of SAV that is within or near the SAV Glycoprotein E1 genomic region (GenBank Acc. No. AY604238) within the SAV genomic sequence;
a pathogen genomic region for the detection of VHSV that is within or near the VHSV Nucleoprotein genomic region (GenBank Acc. No. EF079895) within the VHSV genomic sequence; and
a pathogen genomic region for the detection of Renibacterium salmoninarum that is within or near the Renibacterium salmoninarum Major Soluble Antigen genomic region (GenBank Acc. No. AF123890) within the Renibacterium salmoninarum genomic sequence.

In addition, there is provided a method for detecting the presence or absence of at least one pathogen including: IHNV, IPNV, ISAV, SAV VHSV, or Renibacterium salmoninarum in a sample. The method comprises:
PCR amplification of one or more genomic regions of the at least one pathogen using one or more pair of amplification primers,
target specific primer extension (TSPE) templated on the amplified pathogen genomic region(s) using one or more TSPE primer to produce at least one TSPE primer extension product, and
detection of said TSPE primer extension products, wherein the presence of at least one of the TSPE primer extension products indicates the presence of the at least one pathogen;
wherein the one or more TSPE primers comprise at least one of the following:
a TSPE primer for the detection of IHNV comprising a nucleic acid sequence having at least 80% identity to at least nucleic acids 25 to 44 of SEQ ID NO: 14, or a fragment thereof of at least 15 contiguous nucleotides;
a TSPE primer for the detection of IPNV comprising a nucleic acid sequence having at least 80% identity to at least nucleic acids 25 to 43 of SEQ ID NO: 12, or a fragment thereof of at least 15 contiguous nucleotides;
a TSPE primer for the detection of ISAV comprising a nucleic acid sequence having at least 80% identity to at least nucleic acids 25 to 42 of SEQ ID NO: 1, or a fragment thereof of at least 15 contiguous nucleotides;
a TSPE primer for the detection of SAV comprising a nucleic acid sequence having at least 80% identity to at least nucleic acids 25 to 44 of SEQ ID NO: 15, or a fragment thereof of at least 15 contiguous nucleotides;
a TSPE primer for the detection of VHSV comprising a nucleic acid sequence having at least 80% identity to at least nucleic acids 25 to 42 of SEQ ID NO: 13, or a fragment thereof of at least 15 contiguous nucleotides; and
a TSPE primer for the detection of Renibacterium salmoninarum comprising a nucleic acid sequence having at least 80% identity to at least nucleic acids 25 to 46 of SEQ ID NO: 18, or a fragment thereof of at least 15 contiguous nucleotides.

The above-described methods may be provided in the form of an assay, including a PCR assay, a RT-PCR assay or a multiplexing RT-PCR assay. In addition, a system may also be provided for performing the aforementioned method or assay.

In certain non-limiting embodiments of the above-described methods, two or more of the pathogens may be detected in the sample simultaneously. Moreover, in other embodiments it may be preferred for three, four, five, or all six of the pathogens to be detected in the sample simultaneously. It is also envisioned that additional pathogens may be added to the methods or assays in panels where more than the six mentioned pathogens are tested.

Also provided herein are polynucleotide primers, including primers comprising a nucleic acid sequence having at least 80%, 85%, 95%, or 99% identity to the sequence of SEQ ID NO: 1, 2, 3, 4, 5, 6, 7, 8, 9, 10, 16, or 17, or a fragment thereof of at least 15 contiguous nucleotides.

Also provided herein is a primer pair for the detection of Infectious Hematopoietic Necrosis Virus (IHNV), comprising isolated nucleic acid primers having at least 80% identity to the sequence of SEQ ID NO: 4 and 9, or fragments thereof of at least 15 contiguous nucleotides.

Also provided herein is a primer pair for the detection of Infectious Pancreatic Virus (IPNV), comprising isolated nucleic acid primers having at least 80% identity to the sequence of SEQ ID NO: 2 and 7, or fragments thereof of at least 15 contiguous nucleotides.

Also provided herein is a primer pair for the detection of Infectious Salmon Anemia Virus (ISAV), comprising isolated nucleic acid primers having at least 80%, 85%, 95%, or 99% identity to the sequence of SEQ ID NO: 1 and 6, or fragments thereof of at least 15 contiguous nucleotides.

Also provided is a primer pair for the detection of Salmon Alphaviruses (SAV) comprising isolated nucleic acid primers having at least 80%, 85%, 95%, or 99% identity to the sequence of SEQ ID NO: 5 and 10, or fragments thereof of at least 15 contiguous nucleotides.

Also provided is a primer pair for the detection of Viral Hemorrhagic Septicemia Virus (VHSV), comprising isolated nucleic acid primers having at least 80%, 85%, 95%, or 99% identity to the sequence of SEQ ID NO: 3 and 8, or fragments thereof of at least 15 contiguous nucleotides.

Also provided is a primer pair for the detection of Renibacterium salmoninarum, comprising isolated nucleic acid primers having at least 80%, 85%, 95%, or 99% identity to the sequence of SEQ 11) NO: 16 and 17, or fragments thereof of at least 15 contiguous nucleotides.

Also provided is a use of a polynucleotide primer as described above in the detection of one or more pathogen including: Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), or Renibacterium salmoninarum.

Also provided is a target specific primer extension (TSPE) primer for the detection of Infectious Hematopoietic Necrosis Virus (IHNV), comprising a nucleic acid sequence having at least 80%, 85%, 95%, or 99% identity to the sequence of SEQ ID) NO: 14, or a fragment thereof of at least 15 contiguous nucleotides.

Also provided is a target specific primer extension (TSPE) primer for the detection of Infectious Pancreatic Virus (IPNV), comprising a nucleic acid sequence having at least 80%, 85%, 95%, or 99% identity to the sequence of SEQ ID NO: 12, or a fragment thereof of at least 15 contiguous nucleotides.

Also provided is a target specific primer extension (TSPE) primer for the detection of Infectious Salmon Anemia Virus (ISAV), comprising a nucleic acid sequence having at least 80%, 85%, 95%, or 99% identity to the sequence of SEQ ID NO: 11, or a fragment thereof of at least 15 contiguous nucleotides.

Also provided is a target specific primer extension (TSPE) primer for the detection of Salmon Alphaviruses (SAV), comprising a nucleic acid sequence having at least 80%, 85%, 95%, or 99% identity to the sequence of SEQ ID NO: 15, or a fragment thereof of at least 15 contiguous nucleotides.

Also provided is a target specific primer extension (TSPE) primer for the detection of Viral Hemorrhagic Septicemia Virus (VHSV), comprising a nucleic acid sequence having at least 80%, 85%, 95%, or 99% identity to the sequence of SEQ ID NO: 13, or a ligament thereof of at least 15 contiguous nucleotides.

Also provided is a target specific primer extension (TSPE) primer for the detection of Renibacterium salmoninarum, comprising a nucleic acid sequence having at least 80%, 85%, 95%, or 99% identity to the sequence of SEQ ID NO: 18, or a fragment thereof of at least 15 contiguous nucleotides.

Optionally, the ligaments noted above may all comprise 16, 17, 18, 19 or more contiguous nucleotides of the noted sequences. The primers may be synthetically prepared according to known methods.

Herein, there is also provided the use of a TSPE primer as described above in a method for the detection of one or more pathogen including: Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), or Renibacterium salmoninarum.

In addition, there is further provided a kit for detecting at least one pathogen including: Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), or Renibacterium salmoninarum in a sample, the kit including:
one or more amplification primer or primer pair as above, and/or
one or more TSPE primer as described above.

The assay kit may also comprise instructions for carrying out the methods as described herein.

### BRIEF DESCRIPTION OF THE DRAWINGS

These and other features of the invention will become more apparent from the following description in which reference is made to the appended drawings wherein:

FIG. 1 shows a graphic representation of an embodiment of the present invention, illustrating a multiplex diagnostic assay for detection of salmonid pathogens.

FIG. 2 shows a graphic representation of an embodiment of the present invention, illustrating the nucleic acid PCR operations involved in a multiplex diagnostic assay for detection of salmonid pathogens. In the example shown, ISAV virus is identified in a sample.

FIG. 3 is a flow diagram showing a basic procedure for a multiplex diagnostic assay for detection of salmonid pathogens in accordance with an embodiment of the present invention.

### DETAILED DESCRIPTION

Described herein is a diagnostic assay, as well as various PCR primers and TSPE primers which can be used for the detection of salmonid pathogens. In certain embodiments, these primers and probes can be used in a method capable of simultaneous detection of more than one pathogen in a sample using multiplexing technology.

In certain non-limiting embodiments, the assay is capable of simultaneous detection of two or more salmonid pathogens in a sample, including IHNV, IPNV, ISAV, SAV, VHSV, and Renibacterium salmoninarum. In further non-limiting embodiments, the assay can be customized for specific detection of 1, 2, 3, 4, 5, or 6 pathogens as a singleplex (e.g. ISAV, IHNV, IPNV, SAV, VHSV, or Renibacterium salmoninarum only), duplex (e.g. ISAV and IPNV, or other duplex combinations of IHNV, IPNV, ISAV, SAV, VHSV, and Renibacterium salmoninarum), triplex (e.g. IHNV, ISAV, and VHSV, or other triplex combinations of IHNV, IPNV, ISAV, SAV, VHSV, and Renibacterium salmoninarum), quadruplex (IHNV, ISAV, SHSV, and IPNV, or other quadruplex combinations of IHNV, IPNV, ISAV, SAV, VHSV, and Renibacterium salmoninarum), pentaplex (e.g. ISAV, IPNV, IHNV, SAV, and VHSV, or other pentaplex combinations of IHNV, IPNV, ISAV. SAV, VHSV, and Renibacterium salmoninarum), or hexaplex (e.g. IHNV, IPNV, ISAV, SAV, VHSV, and Renibacterium salmoninarum), depending on the needs of the user.

The assay may be especially useful for disease detection in salmonid stocks during disease outbreaks, and for surveillance and regulatory testing.

Without wishing to be limiting in any way, it is envisioned that some or all of the following steps may be performed when carrying out an assay according to the present invention:

[1] Amplification of viral RNA and/or bacterial genetic material in a sample using multiplex RT-PCR with primer pairs that specifically target each of the salmonid pathogens to be detected (i.e. one or more of ISAV, IPNV, IHNV, SAV, VHSV, Renibacterium salmoninarum, and any others that may be included in the detection panel). The amplified region of each pathogen to be detected is preferably unique to that pathogen, and conserved such that pathogen mutations will not prevent primer annealing.

[2] Carrying out target specific primer extension (TSPE) reactions to prepare labeled (e.g. with biotin or other label) oligonucleotides from the PCR products of the previous step, which may be accomplished using specifically designed tagged TSPE primers (TAG-TSPEs). TAG-TSPEs may have a pathogen-specific primer sequence, and a specifically assigned TAG sequence.

[3] Hybridizing the TSPE reaction products via the TAG-TSPE TAG sequences to fluorescent microbeads containing a corresponding anti-tag sequence (for example, microbeads commercially available from Luminex Corporation). The anti-tag sequence of each fluorescent microbead may be matched to a specific microbead label. For example, a microbead with an anti-TAG sequence specific for an ISAV tag sequence may be labeled with one fluorophore, and a microbead with an anti-TAG sequence for an IPNV tag sequence may be labeled with a different and distinguishable fluorophore. In this way the microbead fluorophore may be used to identify the pathogen specificity of the anti-TAG sequence carried on the microbead.

[4] Reacting the microbead-bound TSPE reaction products, which carry a label (e.g. biotin or other label) as described above, with a reporter (e.g. streptavidin and phycoerythrin, SAPE, or other reporter) which causes fluorescent emission from the biotinylated product.

[5] Detecting the reporter in a detection step. For example, fluorescence from the biotin-SAPE reactions, and the co-localized fluorescence from the labeled microbeads to which the biotinylated TSPE reaction products are bound, may be detected and transmitted into a computer that transforms the emission into numerical values for detecting, interpreting, and evaluating the presence or absence of one or more salmonid pathogens in the sample. By way of example, the Luminex Mag-Pix system may be used for these purposes, in which fluorescence from the SAPE reaction is matched to microbead fluorescence. As described above, microbead fluorescence may be used to determine the pathogen specificity of the microbead anti-TAG sequence, and thus the presence and identity of pathogen in a sample may be identified.

### Example 1

### Detection of ISAV

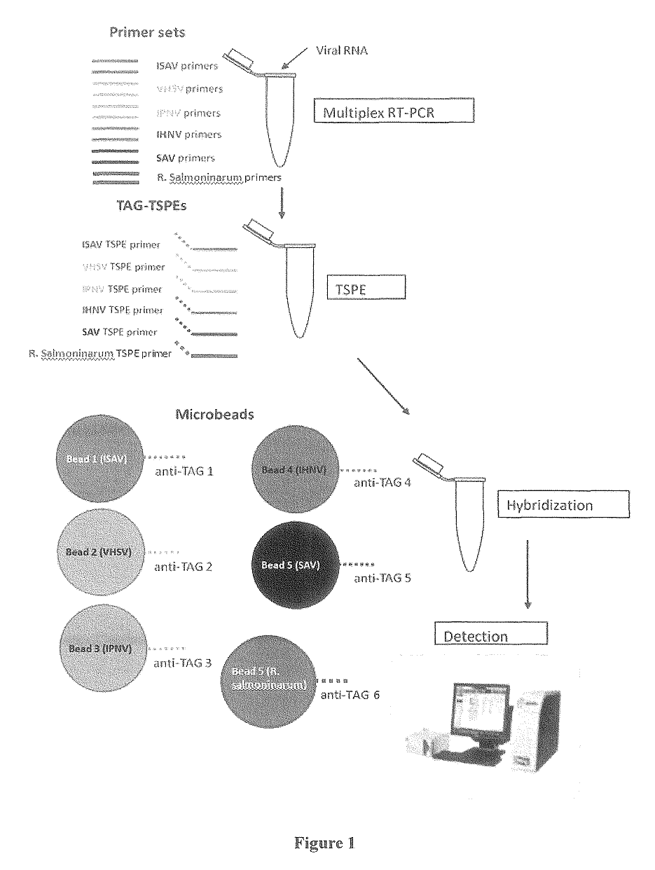

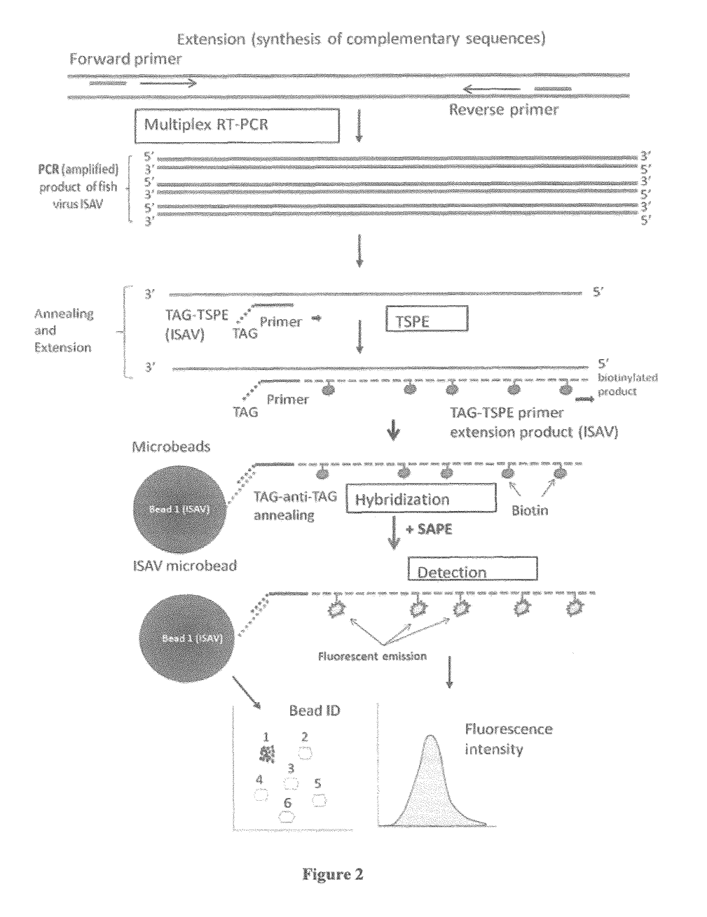

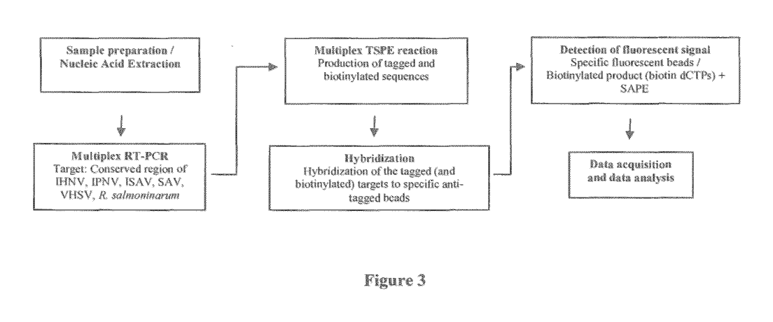

An example of a multiplex diagnostic assay for detection of salmonid pathogens according to an embodiment of the present invention is described in further detail below with reference to FIGS. 1, 2, and 3. FIG. 1 shows addition steps a user may carry out when performing the method. FIG. 2 shows the details of the nucleic acid amplification, annealing, extension and detection steps, and FIG. 3 shows a flow diagram summarizing the steps of the non-limiting example described below. These figures provide an example of a non-limiting embodiment of the present invention, in which a sample is analyzed for the presence of one or more pathogens from ISAV, IPNV, IHNV, SAV, VHSV, and Renibacterium salmoninarum. In this non-limiting example, the sample contains ISAV.

As illustrated, multiplexed real time-PCR is carried out in a first step (Multiplex RT-PCR, shown in FIGS. 1, 2, and 3). FIG. 1 illustrates the addition step for this stage in which primer pairs for detecting up to 6 of the pathogens listed above are added to a sample for multiplexed PCR amplification, and FIG. 2 illustrates the nucleic acid PCR operations that may occur if one or more of the pathogens to be detected is/are present in the sample.

For this initial multiplex PCR step of the present example, target regions within the genomes of each of the 6 pathogens detected by the assay (ISAV, IPNV, IHNV, SAV, VHSV, and Renibacterium salmoninarum) have been selected, and conserved segments within each target region have been identified. In this example, PCR primer pairs with specificity and sensitivity for detecting each of the conserved segments from each pathogen to be detected have been optimized and verified as described below in Example 2. The selection of conserved regions in this example is such that the regions are unique to each pathogen, and remain mostly resistant to pathogen mutation which might otherwise affect detection using the specified PCR primer pair. Table 1 contains PCR primer pairs utilized in this non-limiting example, which have been optimized for the multiplex RT-PCR-based simultaneous detection of one or more pathogens from ISAV, IPNV, IHNV, SAV, VHSV, and Renibacterium salmoninarum.

In a next step of this example, PCR amplified products produced in the multiplex PCR step (in this example, multiplex PCR amplified an ISAV product, as shown in FIG. 2) are subjected to a multiplexed target specific primer extension (TSPE) step, as shown in FIGS. 1, 2, and 3. As shown in FIG. 1, tagged TSPE primers (TAG-TSPE) for detecting each pathogen are added to the products from the previous step. TAG-TSPEs may carry a primer region specific for binding one of the conserved regions used to detect the pathogens, and a TAG region utilized in a subsequent step described below. The nature of the TAG-TSPEs used in this example is further clarified in FIG. 2. The addition of TAG-TSPEs enables a multiplexed TSPE step to be performed using the primer region of the TAG-TSPEs, which may result in TAG-TSPE primer extension if pathogen is present in the sample. Table 2 contains the TAG-TSPE sequences used in this example, which have been optimized for the multiplex PCR-based simultaneous detection of one or more pathogens selected from ISAV, IPNV, IHNV, SAV, VHSV, and Renibacterium salmoninarum as described in Example 2.

The TAG-TSPE primer extension step is used to produce labeled (in the present example biotinylated) TAG-TSPE primer extension products. As shown in the example of FIG. 2, which is not intended to limit the scope of the present invention, the TAG-TSPE primer extension products produced in this step are all derived from ISAV (the only pathogen present in the sample analyzed in this example), and are labeled with biotin using techniques known in the art.

As discussed above, TAG-TSPEs may carry a TAG region, which may be retained in the TAG-TSPE primer extension products produced in the step described above, as shown in FIG. 2. The TAG region of the TAG-TSPEs, much like the primer region of the TAG-TSPEs, may be unique for each pathogen to be detected in the assay. More specifically, the TAG-TSPEs may be designed such that each TAG-TSPE with a primer specific for a particular pathogen carries a TAG sequence that is uniquely assigned to the same particular pathogen.

In a further step of the current example, as shown in FIG. 1, differently labeled microbeads carrying anti-TAG sequences specific for each of the TAG sequences of the TAG-TSPEs are added to the sample. The anti-TAG sequences on the differently labeled microbeads may be selective for one pathogen amplicon per microbead. The nature of the labeled microbeads may be better understood by making reference to the non-limiting example described in FIG. 2. As shown, the biotin labeled TSPE primer extension products in this example carry a TAG sequence specifically assigned to ISAV. Thus, when differently labeled microbeads carrying anti-TAG sequences are added to the sample, the biotin labeled TSPE primer extension products derived from ISAV hybridize to the labeled beads carrying the ISAV anti-TAG sequence. In the present example, each microbead has a known label/anti-TAG identity, such that the label of each microbead can be used to determine the identity of the anti-TAG sequence carried by the microbead.

In a subsequent step of the present example, as shown in FIG. 2, the biotinylated TSPE primer extension products hybridized to the labeled microbeads are reacted with streptavidin and phycoerythrin reporter (SAPE) as is known in the art. The reporter reaction results in the production of fluorescence from biotinylated ISAV TSPE primer extension products, which are hybridized to labeled ISAV anti-TAG microbeads.

In the detection step shown in the example in FIG. 2, SAPE fluorescing microbeads are analyzed to determine the identity of the microbead label. The microbead label reveals the anti-TAG sequence carried by the microbead, and therefore the TAG sequence of the SAPE fluorescing biotinylated TSPE primer extension products bound to the microbead may be identified. In the Example given in FIGS. 1 and 2, fluorescing SAPE reaction is identified in the detection step using a Luminex Mag-Pix™ system, and the label of the labeled microbeads carrying SAPE fluorescing TSPE products matches to an ISAV anti-TAG sequence. Therefore, the assay correctly determines that the sample used in this example contained ISAV virus.

A non-limiting example of an experimental protocol for performing a multiplex diagnostic assay to detect salmonid pathogens may include the following reagents, supplies, equipment, and steps:

List of Reagents and Laboratory Supplies:
1.5 ml copolymer microcentrifuge tubes, natural (USA Scientific, Item No. 1415-2500)
100 mM dNTP set (Invitrogen™, Cat. No. 10297-018)
Biotin-14-dCTP (Invitrogen™, Cat. No. 19518-018)
Bovine Serum Albumin (Fisher BioReagents®, Cat. No. BP9706-100)
Corning™ Costar™ Thermowell™ Plate Cap Strips and Sealing Tape (Cat. No. 07-200-542)
Corning™ Thermowell™ 96-Well PCR Microplates (Cat. No. 07-200-541)
Exo/SAP-IT® (Biolynx®, Prod. No. UB78201)
MagPlex®-TAG™ microspheres (Luminex®, Prod. No. MTAG-A012, -A013, -A014, -A015, -A021, -A028)
Micropipettes and tips
Microtubes (1.5, 0.5 & 0.2 mL)
One Step RT-PCR kit (QIAGEN®, Cat. No. 210212)
PCR and TSPE Primers (IDT®)
Platinum® Taq DNA polymerase (Invitrogen™, Cat. No. 10966-034)
Streptavidin-R-phycoerythrin conjugate SAPE (Invitrogen™, Cat. No. S-866)
xTAG® 10× buffer (Luminex®, Prod. No. GR001C0060)

List of Equipment:
Centrifuge (Eppendorf, Cat. No. 5430R)
Compact Ultrasonic cleaner Sonicator (Cole-Palmer®, OF-08849-00)
Mag Pix analyzer (Luminex®)
Thermo cycler GeneAmp PCR system 9700 96 well silver (Life Technologies™, Cat. No. N8050001)
Vortex mixer (Fisher®, Cat. No. 02215365)

A. Multiplex RT-PCR

1. Using QIAGEN® One Step RT-PCR kit, prepare a master mix in a total volume of 50 μL per reaction. The master mix also contains a primer pool (0.4 μM) with a primer set for each of the six pathogens.

2. Transfer aliquots of 5 μL of RNA/genomic material extracted from each pathogen in tissue culture to each reaction microtube.

3. Put the microtubes in a thermo cycler under the following conditions for the RT-PCR reaction: reverse transcription 30 min/50° C., initial PCR activation 15 min/95° C., 35 cycles of denaturation 1 min/94° C., annealing 45 sec/58° C., and extension 1 min/72° C., followed by a step of final extension 10 min/72° C.

4. Visualize the PCR products in a 1.5% agarose gel through a UV transilluminator (optional).

B. Exo-SAP-IT Treatment

5. Treat the PCR products by mixing 7.5 μL of them with 3 μL of Exo/SAP-IT (Biolynx®).

6. Incubate at 37° C. for 30 minutes and inactivate Exo/SAP-IT by heating to 80° C. for 15 minutes. Hold the treated reactions at 4° C. (steps can be performed in a thermal cycler).

C. Multiplex TSPE Reaction

7. Prepare a Master mix containing 2 μL of 10×TSPE reaction buffer, 0.5 μL of 50 mM MgCl₂, 1 μL of 20× (500 nM each) Tag-TSPE primer mix, 0.15 μL of 5 U/μL Taq DNA polymerase, 1 μL of 20× (100 μM each) dNTP mix (except dCTP), 0.25 μL of 400 μM biotin-dCTP, and 10.1 μL of dH₂O, per sample.

Note: All these reagents are from Invitrogen®.

8. Transfer aliquots of 15 μL of Master mix to 200 μL microtubes, and add 5 μL of treated PCR reaction (sample).

9. Put the microtubes in a thermocycler under the following cycling conditions: 96° C./2 min; 30 cycles of 94° C./30 sec, 55° C./1 min and 74° C./2 min.

D. Hybridization to MagPlex-TAG Microspheres

10. Select the 5 microsphere sets (Luminex®) from storage at 4° C. to room temperature. Resuspend them by vortexing (30 sec) and sonication (1 min).

11. Transfer 41.6 μL from each set into a 1.5 mL microtube. This tube will have a mixture of 520000 beads in 208 μL.

12. Centrifuge at ≧8000×g for 1-2 min, and remove the supernatant (being careful not to disturb the pellet).

13. Resuspend the pellet with 1 mL of 1.1×Tm hybridization buffer. Vortex (10 sec) and sonicate (10 sec) twice. This stock solution will contain 520000 beads mix/mL or 104000 bead set/mL (104 bead set/μL).

14. Transfer 24 μL of this solution to each well of a microplate.

15. Add 1 μL of dH₂O to each background well or 1 μL of TSPE reactions to appropriate wells. Cover the plate to prevent evaporation.

16. Denature at 96° C. for 90 sec and hybridize at 37° C. for 30 min (thermocycler).

17. Prepare reporter mix by diluting streptavidin-R-phycoerythrin (Invitrogen®) to 5 μg/mL in 1×Tm hybridization buffer with 0.1% BSA.

18. Add 100 μL of reporter mix to each well. Mixing gently.

19. Incubate at 37° C. for 15 min.

20. Analyze 50 μL at 37° C. on the Luminex Mag Pix analyzer.

Note: The block should be pre-heated and the protocol created in the analyzer should include a wash step before reading.

The non-limiting example provided above is for demonstrative purposes only, and is in no way intended to limit the scope of the present invention.

### Example 2

### Optimized Primers and TAG-TSPEs

As described above, the multiplex diagnostic assay for salmonid pathogens described herein may employ multiple primer sets and TAG-TSPE primers in multiplex PCR steps. The assay may benefit from high specificity (a primer set for PCR amplification of a target region in one pathogen genome should not PCR amplify sequence from any other pathogen in the assay), high sensitivity (low concentrations of pathogen in the sample should be detectable), and compatibility between primer sets (and TAG-TSPEs) during multiplex PCR, which may simultaneously amplify more than one sequence. Optimized pathogen-specific primer sets utilized in the previously described example are shown in Table 1, and optimized TAG-TSPE primers in Table 2. Genomic segments/genes targeted by the primers shown in Tables 1 and 2 are outlined in Table 3.

TABLE 1
Sequences of PCR primer pairs (5′ to 3′) optimized for the multiplex RT-PCR-based
simultaneous detection of two or more pathogens selected from ISAV, IPNV, IHNV, SAV,
VHSV, and Renibacteritun salmoninarum.

| RT-PCR Primer | Primer Forward | Seq ID | Primer Reverse | Seq ID | Product |
|---|---|---|---|---|---|
| Pool | Sequence | No. | Sequence | No. | (bp) |
| ISAV-Seg8 | GGC AAT GGT GTA TGG TAT GA | 1 | GAT CTT GTC GTC CAG CTC | 6 | 276 |
| 361F/619R | | | | | |
| IPNV-VP2 | CAA CAG TGA CGG GAG ACA | 2 | CTC ATA GGC CAC CAG TGT | 7 | 362 |
| 637F/981R | | | | | |
| VHSV-N | GAC AGG AAT GAC CAT GAT TG | 3 | GAG TCC ACT GCG TAC TTC | 8 | 285 |
| 660F/927R | | | | | |
| IHNV-G | ACT CCA TAC CTC CTA TCCA | 4 | GCT CAT CGG TTC CAT CAT | 9 | 294 |
| 970F/1246R | | | | | |
| SAV-E1 | GAC CTC AAG ATC GTG GCT | 5 | GCA CTG ATC TTA CAA CCG T | 10 | 326 |
| 10866F/11173R | | | | | |
| Rs-msa1 | GGT GGC TCT TAT AGT TCT GG | 16 | GGC AGG ACC ATC TTT GTT AT | 17 | 135 |
| 493F/608R | | | | | |

TABLE 2
TAG-TSPE primers (5′ to 3′) optimized for the multiplex
PCR-based simultaneous detection of two or more pathogens
selected from ISAV, IPNV, IHNV, SAV, and VHSV, and
Renibacterium salmoninarum.

| TSPE-Primer | TSPS-primer | Seq ID | Product |
|---|---|---|---|
| Mix | sequence (tag + primer) | No. | (bp) |
| T-ISAV-Seg8 | CAT AAT CAA TTT CAA CTT TCT ACT | 11 | 170 |
| 513R | CCG GAA GTC GAT GAA CTG | | |
| T-IPNV-VP2 | CAA ATA CAT AAT CTT ACA TTC ACT | 12 | 211 |
| 788F | AGG AGT CTC AGC CAA GAT G | | |
| T-VHSV-N | TAC TTC TTT ACT ACA ATT TAC AAC | 13 | 193 |
| 835R | CAG CAT CCG ACT CAT CAT | | |
| T-IHNV-G | TCA AAC TCT CAA TTC TTA CTT AAT | 14 | 239 |
| 1189R | GGA ATA ATG GTG GTG TTG TT | | |
| T-SAV-E1 | CAC TTA ATT CAT TCT AAA TCT ATC | 15 | 188 |
| 11004F | CAA GCT AGG TCA ACC AAC TA | | |
| T-Rs-msa1 | [symbol] | 18 | 82 |
| 546F | TCT TGT TGT TCC CGT AGT AAA C | | |

[symbol]

indicates data missing or illegible when filed

TABLE 3
Genomic sequences targeted by PCR and TSPE Primers shown in Tables 1 and 2.

| | | | | | | Position of |
|---|---|---|---|---|---|---|
| | | | | Position of first | | first and last |
| | | | GenBank Acc. No. | and last | | nucleotide of |
| | | Number | of representative | nucleotide of | Primer | amplified |
| Target | | (Tables | sequence of the | primers in target | length | segment & |
| gene/segment | Primer | 1&2) | genomic target | sequence | (bp) | length (bp) |
| IHNV- | PCR- | 4 | AY331666 | 970-988 | 19 | 970-1263 |
| Glycoprotein | 970F | | | | | (294) |
| | PCR- | 9 | | 1246-1263 | 18 | |
| | 1246R | | | | | |
| | TSPF- | 14 | | 1189-1208 | 20 | 970-1208 |
| | 1189R | | | | | (239) |
| IPNV-Major | PCR- | 2 | FN257526 | 637-654 | 18 | 637-998 |
| capsid | 637F | | | | | (362) |
| polypeptide VP2 | PCR- | 7 | | 981-998 | 18 | |
| | 981R | | | | | |
| | TSPE- | 12 | | 788-806 | 19 | 788-998 |
| | 788F | | | | | (211) |
| ISAV-Segment 8 | PCR- | 1 | AF404340 | 361-380 | 20 | 361-636 |
| | 361F | | | | | (276) |
| | PCR- | 6 | | 619-636 | 18 | |
| | 619R | | | | | |
| | TSPE- | 11 | | 513-530 | 18 | 361-530 |
| | 513R | | | | | (170) |
| SAV- | PCR- | 5 | AY604238 | 10866-10883 | 18 | 10866-11191 |
| Glycoprotein E1 | 10866F | | | | | (326) |
| | PCR- | 10 | | 11173-11191 | 19 | |
| | 11173R | | | | | |
| | TSPE- | 15 | | 11004-11023 | 20 | 11004-11191 |
| | 11004F | | | | | (188) |
| VHSV- | PCR- | 3 | EF079895 | 660-678 | 20 | 660-944 |
| Nucleoprotein | 660F | | | | | (285) |
| | PCR- | 8 | | 927-944 | 18 | |
| | 927R | | | | | |
| | TSPE- | 13 | | 835-852 | 18 | 660-852 |
| | 835R | | | | | (193) |
| Rs-Major | PCR- | 16 | AF123890 | 493-512 | 20 | 493-627 |
| soluble antigen | 493F | | | | | (135) |
| | PCR- | 17 | | 608-627 | 20 | |
| | 608R | | | | | |
| | TSPE- | 18 | | 546-567 | 22 | 546-627 |
| | 546F | | | | | (82) |

Note:
The PCR or TSPE reverse primer 5′-3′ sequences given in Tables 1 and 2 correspond to the reverse complement of the target genomic sequences determined by the first and last nucleotide.

The performance of a multiplex diagnostic assay for salmonid pathogens employing the primer sets and TAG-TSPEs shown in Tables 1 and 2 (and discussed in Example 1) has been verified experimentally. Table 4 shows the results of experimentation designed to determine the specificity of the exemplified assay. In these experiments, the extracted nucleic acid of each of the ISAV, IPNV, VHSV, IHNV, SAV, and Renibacterium salmoninarum pathogens was subjected to the exemplified assay for detecting one or more of the 6 pathogens. As shown in Table 4, in each case the assay correctly detected only the pathogen present in the sample, and indicated an absence of any of the other 5 pathogens. These results demonstrate the specificity of the assay.

TABLE 4
Optimization of the core multiplex assay for
salmonid pathogens - specificity

| | Results - Core Multiplex Assay for Salmonid Pathogens | | | | | |
|---|---|---|---|---|---|---|
| Name of | | | | | | R. |
| pathogen | ISAV | IPNV | VHSV | IHNV | SAV | salmoninarum |
| ISAV | + | − | − | − | − | − |
| IPNV | − | + | − | − | − | − |
| VHSV | − | − | + | − | − | − |
| IHNV | − | − | − | + | − | − |
| SAV | − | − | − | − | + | − |
| R. | − | − | − | − | − | + |
| salmoninarum | | | | | | |

Table 5 provides experimental results related to the sensitivity of the multiplex assay for salmonid pathogens provided herein. Several 10-fold dilutions of specific virus preparations from the Regional Diagnostic Virology Services (RDVS) at AVC-UPEI were used in the comparative sensitivity experiments, which compare the sensitivity of the present example to assays employing the PCR design and methodologies currently being utilized in the field. As shown in the results presented in Table 5, the present example has remarkable sensitivity compared to the existing molecular diagnostic assays being utilized. For the 5 viruses being detected in the comparative sensitivity experiments shown in Table 5, the multiplex assay of the present example was consistently able to detect virus at lower concentrations than the other assays in the comparison study. The sensitivity of the core multiplex diagnostic assay for salmonid viruses of the present example is similar in sensitivity to the gold standard, which is Virus Isolation.

TABLE 5
Optimization of the core multiplex assay for salmonid pathogens - sensitivity

| Dilution | ISAV | | IPNV | | VHSV | | |
|---|---|---|---|---|---|---|---|
| (Specific virus | Multiplex | Traditional | Multiplex | Traditional | Multiplex | Traditional Nested PCR | |
| stock) | Assay | RT-PCR | Assay | RT-PCR | Assay | 1° RT-PCR | 2° PCR |
| 10⁻¹ | + | + | + | + | + | − | + |
| 10⁻² | + | + | + | + | + | − | + |
| 10⁻³ | + | − | + | − | + | − | − |
| 10⁻⁴ | + | − | − | − | + | − | − |
| 10⁻⁵ | + | − | − | − | + | − | − |
| 10⁻⁶ | − | − | − | − | + | − | − |
| 10⁻⁷ | − | − | − | − | − | − | − |

| | IHNV | | | SAV | |
|---|---|---|---|---|---|
| Dilution | | Traditional Nested PCR | | | Real time |
| (Specific virus stock) | Multiplex Assay | 1° RT-PCR | 2° PCR | Multiplex Assay | PCR |
| 10⁻¹ | + | − | − | + | + |
| 10⁻² | − | − | − | + | − |
| 10⁻³ | − | − | + | + | + |
| 10⁻⁴ | + | − | + | + | + |
| 10⁻⁵ | − | − | + | + | − |
| 10⁻⁶ | − | − | − | + | − |
| 10⁻⁷ | − | − | − | − | − |

Renibacterium salmoninarum

| | | | | Köningsson et al., | |
|---|---|---|---|---|---|
| | | OIE 2000 | OIE 2003 | 2005 (Fluorescent | Multiplex Assay |
| DNA quantity | | (PCR)¹ | (nested PCR)² | PCR)³ | (6-plex) |
| 100 | ng | + | + | + | + |
| 10 | ng | + | + | + | + |
| 1 | ng | + | + | + | + |
| 0.1 | ng | + | − | + | + |
| 10 | pg | − | − | + | + |
| 1 | pg | − | − | + | + |
| 0.1 | pg | − | − | + | + |

¹OIE “Manual of Diagnostic Tests for Aquatic Animals”, Bacterial Kidney Disease (Renibacterium salmoninarum), 2000.
²OIE “Manual of Diagnostic Tests for Aquatic Animals”, Bacterial Kidney Disease (Renibacterium salmoninarum), 2003.
³Köningsson, M. H.; Ballagi, A.; Jansson, E.: Johansson, K-E. “Detection of Renibacterium salmoninarum in tissue samples by sequence capture and fluorescent PCR based on the 16S rRNA gene”. Veterinary Microbiology 105: 235-243, 2005.

In summary, the example of the multiplex assay described in the examples is useful for the simultaneous detection of salmonid pathogens in a sample. The examples illustrated herein provide 6 primer pairs that are especially useful for simultaneous detection of up to 6 pathogens (ISAV, IPNV, VHSV, IHNV, SAV, and Renibacterium salmoninarum), and have been optimized for pathogen specificity and sensitivity in a multiplex PCR-based assay. These 6 primer pairs were designed to amplify specific regions within the genomes of each of the 6 pathogens. The amplification regions of the described examples have been selected on the basis of resistance to mutation, uniqueness to each pathogen, compatibility with multiplex PCR amplification, and proportion of the 4 nucleotides in the sequence. The proportion of the 4 nucleotides, which is related to the quantity of labeled (e.g. biotinylated) nucleotide that may be produced during the TSPE reaction, may be related to the sensitivity of the assay. The description illustrated herein also provides TAG-TSPE primer sequences and tag sequences for simultaneous detection of one or more of the 6 pathogens (ISAV, IPNV, VHSV, IHNV, SAV, and Renibacterium salmoninarum), and which are compatible with the amplification regions and multiplex PCR steps of the assay described in the examples above.

One or more currently preferred embodiments have been described by way of example. It will be apparent to persons skilled in the art that a number of variations and modifications can be made without departing from the scope of the invention as defined in the claims.

1. A method for detecting the presence or absence of at least one pathogen selected from: Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV) or Renibacterium salmoninarum in a sample, said method comprising:
PCR amplification of one or more genomic region of said at least one pathogen using one or more pair of amplification primers,
target specific primer extension (TSPE) templated on said amplified pathogen genomic region(s) using one or more TSPE primer to produce at least one TSPE primer extension product, and
detection of said at least one TSPE primer extension product, wherein the presence of at least one of said at least one TSPE primer extension product indicates the presence of said at least one pathogen;
wherein said one or more pair of primers comprise at least one of the following:
at least one primer for the detection of IHNV selected from an isolated nucleic acid having at least 80% identity to the sequence of SEQ ID NO: 4 or 9, or fragments thereof of at least 15 contiguous nucleotides;
at least one primer for the detection of IPNV, selected from an isolated nucleic acid having at least 80% identity to the sequence of SEQ ID NO: 2 or 7, or fragments thereof of at least 15 contiguous nucleotides;
at least one primer for the detection of ISAV, selected from an isolated nucleic acid having at least 80% identity to the sequence of SEQ ID NO: 1 or 6, or fragments thereof of at least 15 contiguous nucleotides;
at least one primer for the detection of SAV, selected from an isolated nucleic acid having at least 80% identity to the sequence of SEQ ID NO: 5 or 10, or fragments thereof of at least 15 contiguous nucleotides;
at least one primer for the detection of VHSV, selected from an isolated nucleic acid having at least 80% identity to the sequence of SEQ ID NO: 3 or 8, or fragments thereof of at least 15 contiguous nucleotides; and
at least one primer for the detection of Renibacterium salmoninarum, selected from an isolated nucleic acid having at least 80% identity to the sequence of SEQ ID NO: 16 or 17, or fragments thereof of at least 15 contiguous nucleotides.

2. A method according to claim 1, wherein the presence or absence of two or more of said pathogens is detected in said sample simultaneously.

3. A method according to claim 1, wherein the presence or absence of all six of said pathogens is detected in said sample simultaneously.

4. A polynucleotide primer comprising a nucleic acid sequence having at least 80% identity to the sequence of SEQ ID NO: 1, 2, 3, 4, 5, 6, 7, 8, 9, 10, 16, or 17, or a fragment thereof of at least 15 contiguous nucleotides.

5. The polynucleotide primer of claim 4, wherein said fragment comprises at least 18 contiguous nucleotides of SEQ ID NO: 1, 2, 3, 4, 5, 6, 7, 8, 9, 10, 16, or 17.

6. A primer pair for the detection of:
Infectious Hematopoietic Necrosis Virus (IHNV), comprising isolated nucleic acid primers having at least 80% identity to the sequence of SEQ ID NO: 4 and 9, or fragments thereof of at least 15 contiguous nucleotides;
Infectious Pancreatic Virus (IPNV), comprising isolated nucleic acid primers having at least 80% identity to the sequence of SEQ ID NO: 2 and 7, or fragments thereof of at least 15 contiguous nucleotides;
Infectious Salmon Anemia Virus (ISAV), comprising isolated nucleic acid primers having at least 80% identity to the sequence of SEQ ID NO: 1 and 6, or fragments thereof of at least 15 contiguous nucleotides;
Salmon Alphaviruses (SAV) comprising isolated nucleic acid primers having at least 80% identity to the sequence of SEQ ID NO: 5 and 10, or fragments thereof of at least 15 contiguous nucleotides;
Viral Hemorrhagic Septicemia Virus (VHSV), comprising isolated nucleic acid primers having at least 80% identity to the sequence of SEQ ID NO: 3 and 8, or fragments thereof of at least 15 contiguous nucleotides; or
Renibacterium salmoninarum, comprising isolated nucleic acid primers having at least 80% identity to the sequence of SEQ ID NO: 16 and 17, or fragments thereof of at least 15 contiguous nucleotides.

7.-12. (canceled)

13. A target specific primer extension (TSPE) primer, wherein the TSPE is:
for the detection of Infectious Hematopoietic Necrosis Virus (IHNV), comprising a nucleic acid sequence having at least 80% identity to at least nucleic acids 25 to 44 of SEQ ID NO: 14, or a fragment thereof of at least 15 contiguous nucleotides;
for the detection of Infectious Pancreatic Virus (IPNV), comprising a nucleic acid sequence having at least 80% identity to at least nucleic acids 25 to 43 of SEQ ID NO: 12, or a fragment thereof of at least 15 contiguous nucleotides;
for the detection of Infectious Salmon Anemia Virus (ISAV), comprising a nucleic acid sequence having at least 80% identity to at least nucleic acids 25 to 42 of SEQ ID NO: 11, or a fragment thereof of at least 15 contiguous nucleotides;
for the detection of Salmon Alphaviruses (SAV), comprising a nucleic acid sequence having at least 80% identity to at least nucleic acids 25 to 44 of SEQ ID NO: 15, or a fragment thereof of at least 15 contiguous nucleotides;
for the detection of Viral Hemorrhagic Septicemia Virus (VHSV), comprising a nucleic acid sequence having at least 80% identity to at least nucleic acids 25 to 42 of SEQ ID NO: 13, or a fragment thereof of at least 15 contiguous nucleotides; or
for the detection of Renibacterium salmoninarum, comprising a nucleic acid sequence having at least 80% identity to at least nucleic acids 25 to 46 of SEQ ID NO: 18, or a fragment thereof of at least 15 contiguous nucleotides.

14.-19. (canceled)

20. A kit for detecting at least one pathogen selected from: Infectious Hematopoietic Necrosis Virus (IHNV), Infectious Pancreatic Virus (IPNV), Infectious Salmon Anemia Virus (ISAV), Salmon Alphaviruses (SAV), Viral Hemorrhagic Septicemia Virus (VHSV), or Renibacterium salmoninarum in a sample, said kit comprising:
one or more primer or primer pair as defined in claim 4, and/or
one or more TSPE primer as defined in claim 13.

21. The kit of claim 20, further comprising instructions for carrying out the method of claim 1.

22.-23. (canceled)


SEQUENCE LISTING (18 sequences)

SEQ ID NO: 1 - protein, 20 aa, Artificial Sequence
  ISAV-Seg8 Primer Forward Sequence
GGCAATGGTGTATGGTATGA

SEQ ID NO: 2 - protein, 18 aa, Artificial Sequence
  IPNV-VP2 Primer Forward Sequence
CAACAGTGACGGGAGACA

SEQ ID NO: 3 - protein, 20 aa, Artificial Sequence
  VHSV-N Primer Forward Sequence
GACAGGAATGACCATGATTG

SEQ ID NO: 4 - protein, 19 aa, Artificial Sequence
  IHNV-G Primer Forward Sequence
ACTCCATACCTCCTATCCA

SEQ ID NO: 5 - protein, 18 aa, Artificial Sequence
  SAV-E1 Primer Forward Sequence
GACCTCAAGATCGTGGCT

SEQ ID NO: 6 - protein, 18 aa, Artificial Sequence
  ISAV-Seg8 Primer Reverse Sequence
GATCTTGTCGTCCAGCTC

SEQ ID NO: 7 - protein, 18 aa, Artificial Sequence
  IPNV-VP2 Primer Reverse Sequence
CTCATAGGCCACCAGTGT

SEQ ID NO: 8 - protein, 18 aa, Artificial Sequence
  VHSV-N  Primer Reverse Sequence
GAGTCCACTGCGTACTTC

SEQ ID NO: 9 - protein, 18 aa, Artificial Sequence
  IHNV-G Primer Reverse Sequence
GCTCATCGGTTCCATCAT

SEQ ID NO: 10 - protein, 19 aa, Artificial Sequence
  SAV-E1 Primer Reverse Sequence
GCACTGATCTTACAACCGT

SEQ ID NO: 11 - protein, 42 aa, Artificial Sequence
  T-ISAV-Seg8 TSPE-primer sequence
CATAATCAATTTCAACTTTCTACTCCGGAAGTCGATGAACTG

SEQ ID NO: 12 - protein, 43 aa, Artificial Sequence
  T-IPNV-VP2 TSPE-primer sequence
CAAATACATAATCTTACATTCACTAGGAGTCTCAGCCAAGATG

SEQ ID NO: 13 - protein, 42 aa, Artificial Sequence
  T-VHSV-N TSPE-primer sequence
TACTTCTTTACTACAATTTACAACCAGCATCCGACTCATCAT

SEQ ID NO: 14 - protein, 44 aa, Artificial Sequence
  T-IHNV-G TSPE-primer sequence
TCAAACTCTCAATTCTTACTTAATGGAATAATGGTGGTGTTGTT

SEQ ID NO: 15 - protein, 44 aa, Artificial Sequence
  T-SAV-E1 TSPE-primer sequence
CACTTAATTCATTCTAAATCTATCCAAGCTAGGTCAACCAACTA

SEQ ID NO: 16 - protein, 20 aa, Artificial Sequence
  Rs-msa1 Primer Forward Sequence
GGTGGCTCTTATAGTTCTGG

SEQ ID NO: 17 - protein, 20 aa, Artificial Sequence
  Rs-msa1 Primer Reverse Sequence
GGCAGGACCATCTTTGTTAT

SEQ ID NO: 18 - protein, 46 aa, Artificial Sequence
  T-Rs-msa1 TSPE-primer sequence
AATTTCTTCTCTTTCTTTCACAATTCTTGTTGTTCCCGTAGTAAAC